(19) United States
(12) Patent Application Publication
(10) Pub. No.: US 2007/0149882 A1
(43) Pub. Date: Jun. 28, 2007

(54) Device for visualizing object attributes

(76) Inventor: Matthias Wedel, Nurnberg (DE)

Correspondence Address: BRINKS HOFER GILSON & LIONE, P.O. BOX 10395, CHICAGO, IL, 60610, US

(21) Appl. No.: 11/636,057

(22) Filed: Dec. 8, 2006

(30) Foreign Application Priority Data
Dec. 16, 2005 (DE) DE 102005060311.4

(51) Int. Cl. A61B 5/00; G01J 3/30

(52) U.S. Cl. 600/476; 356/317; 356/600; 356/601

(57) ABSTRACT

A device for visualizing object attributes is provided. The visualization device includes a projector that is operative to project in a position-directed manner. A data input device is operative to receive position data and associated position-specific object attribute information. A control device is connected at the input end to the data input device and at the output end to the projector. The control device is operative to control the projector to project a visualization of position-specific object attribute information received from the data input.

The present patent document claims the benefit of the filing date of DE 10 2005 060 311.4 filed Dec. 16, 2005.

### BACKGROUND

The present embodiments relate to a device for visualizing object attributes and to a scanning device for scanning object attributes with the visualization device.

Scanning devices are generally used to scan object attributes. Scanning devices are used to examine, for example, surface characteristics such as roughness, absorption behavior or transparency, optical attributes that are difficult or even impossible for the human eye to perceive, mechanical attributes such as fractures, or material properties such as deposits.

Alternatively, scanning devices are used to detect fluorescence phenomena resulting from excitation with light of a suitable wavelength. For medical purposes, pathological tissue, for example, cancer, may be marked with specific contrast agents that have special fluorescence properties. It is possible to detect said agents by recording fluorescence light emitted by the marked tissue. When the fluorescence light intensity is too low or lies in a wavelength range that cannot be perceived by the human eye, an additional visualization of the fluorescing tissue areas is necessary. Night vision systems are used to view optical attributes that are scarcely perceptible to the human eye due to the low light intensity. Night vision systems scan the optical attributes of an object and illustrate or visualize the object in a form that is perceptible to the human eye.

The information captured by scanning is typically illustrated or visualized by monitor screens or displays. The optical information that is to be displayed may be recorded by an electronic camera and/or a scanning lens system specifically adapted to the respective examination purpose. The scanning lens system operates with electromagnetic radiation, which can lie either in the range of visible light or in other wavelength ranges.

The optical information relating to the scanned object and the information acquired as a result of the scanning is presented together on the display. This joint presentation, of the optical information and the scanning information, enables a user to find his/her bearings in relation to the real object scanned and then to transfer the scanning information related to the real object.

Mentally transferring the information shown on the monitor screen or display to the real scenario is not such a straightforward process. For example, mentally transferring the information from the monitor to the real scenario is difficult since visual points of reference for the transfer are missing when the scenario exhibits poor contrast or is lacking in detail. A user must frequently switch back and forth between the screen and real scenario. A size comparison is also difficult on occasion due to the imaging scale on the screen. Additional orientation problems arise if the scanned surface is uniformly structured and only sections thereof are shown on the screen. Relocating the displayed section on the real surface is then particularly difficult.

The difficulties in the mental transfer are further increased if the scanning device is used directly on the surface that is to be scanned. For example, when sampling of fluorescence light for the purpose of detecting pathological tissue, the scanning device is generally used directly on the surface of the object. Due to the low fluorescence light intensity and the relatively high proportions of diffuse scattered light, the scanning device is used in close proximity to the tissue in order to obtain a sharp, high-resolution scanning image. High image quality and a high resolution are used in, for example, a therapeutic intervention. The scanning image is used to plan the therapeutic intervention.

Generally, a lens system is used to record the visual image of the object with sufficient sharpness. A lens system is also used to image (illustrate) the scanning information, depending on the scanning wavelength. The same lens system may be used for the visual image and the scanning information. An imaging lens system has only a limited depth of field, so a specific distance between the scanning device and the object to be scanned is maintained in order to obtain a sharp image.

Generally, the observer cannot observe the object continuously nor receive a precise real-time impression of the object. When a change in the viewing direction of the observer between object and screen occurs, the observer receives a different impression of the object. For example, an operator must in each case perform an operational step, then direct his/her view onto the screen in order to be able to check the success of the operational step, then once again look at the area of operation in order to perform the next operational step, etc. If the operator is in this case performing a medical intervention on living tissue, then a natural movement of said tissue can act as an additional factor exacerbating the difficulties even further.

### SUMMARY

The present embodiments may obviate one or more of the limitations of the related art. For example, in one embodiment, a visualization device has a projector embodied to project in a position-directed manner. A data input device is embodied to receive position data and/or associated position-specific object attribute information. A control device is connected at the input end to the data input and at the output end to the projector and is embodied to control the projector in such a way that the projector projects, in a position-directed manner, a visualization of position-specific object attribute information received from the data input.

In another embodiment, a scanning device includes a visualization device. The scanning device includes a primary radiation source. A secondary radiation detector is embodied to detect secondary radiation generated by an object due to the incidence of primary radiation. An evaluation device determines position data and associated position-specific object attribute information as a function of a detection by the secondary radiation detector and transmits said position data and associated information to the data input of the visualization device.

In one embodiment, object attribute information, which relates to a specific position at or on the object, may be projected onto the object with direct spatial reference to the specific position. The object attribute information may be projected precisely onto that specific position at which a questionable object attribute was determined, depending on the type of visualization. An object attribute is to be understood as, for example, a visible or invisible optical attribute, a surface characteristic, a material property, an absorption behavior or a transparency, a material property such as a fracture or a deposit, a fluorescence phenomenon or any other suitable attribute that may be determined by scanning.

In one embodiment, the combination of the visualization device with a scanning device achieves the close temporal and spatial correlation of scanning and visualization. In this embodiment, precision and position resolution are increased and a real-time behavior may be guaranteed. A primary radiation source of the scanning device is understood to be, for example, a radiation source. In one embodiment, the radiation source may generate any beam suitable for scanning, for example, a light source in the visible or invisible wavelength range, a laser, an electron beam source, some other particle beam source, or any other suitable radiation source.

In one embodiment, the projector includes a laser display projector. A laser display projector is to be understood to mean that the projector includes a laser radiation source, which projects a laser beam onto a deflection device including, for example, micromirrors. The deflection device is controlled in such a way that the laser beam strikes the projection area, where it generates the desired visualization. Laser radiation source and deflection mirror(s) are controlled by the control device. In one embodiment, the laser beam may be, for example, an individual, monochrome laser beam or an individual laser beam colored by corresponding color filters or a plurality of different colored laser beams. In one embodiment, an individual micromirror or a plurality of micromirrors in a micromirror array may be used depending on the projection system.

In one embodiment, a laser display projector is structurally embodied according to specific requirements so as to be energy-saving and have exceptional light intensity. High light intensity is especially important, particularly in applications under daylight conditions.

In one embodiment, the visualization device includes a numeric or alphanumeric display. The object attributes to be visualized may then be displayed for a user in plaintext.

In one embodiment, the visualization device includes a graphical display of contours. In this embodiment, contrasts that are difficult to detect optically or, for example, changes in material property that are impossible to perceive optically may be displayed directly on the object. In one embodiment, the necessary points of reference (“landmarks”) that enable the mental transfer from a conventional separate display onto the object are completely missing. In this embodiment, the visualization directly on the object is beneficial when object attribute limits cannot be perceived at all.

A further advantageous exemplary embodiment provides that the visualization includes a graphical display of areas. This will enable areas with certain object attributes, e.g. material properties, to be represented directly on the object as self-contained areas and consequently particularly realistically. A correspondingly two-dimensional visualization is realistic and therefore particularly intuitive and easy for the user to register in particular in the case of fluorescence detection in which it is possible that areas of tissue marked with contrast agent are detected.

In one embodiment, a scanning device includes a visualization device. The scanning device scans object attribute information, which includes, for example, a contrast in the visible and/or invisible wavelength range, a surface characteristic, an absorption behavior, a transparency, a fracture and/or a deposit. The scanned object attribute information may also include the occurrence of a fluorescence. In this embodiment, it may be possible to capture, and then also to visualize, the object attribute information using a common, integrated device. The integrated solution, for example, simplifies the use of position data to a particular degree, since the position of scanning device and visualization device relative to one another are known. From the known spatial association with respect to one another it is then possible, for example, when the scanning device determines position data, to convert from the position data directly to the associated relative position in relation to the visualization device. In this embodiment, the visualization will, for example, if desired, actually be projected precisely at the location with which the visualized object attribute information is associated. For example, a contour may be displayed precisely at the location of the material property limit.

In one embodiment, the projection position-is simple and error-resistant when the scanning device and visualization device are arranged in close proximity to one another and with substantially coinciding projection directions.

In one embodiment, the visualization device includes a visualization in real time. A visualization in real time is a projection of the visualization that follows immediately in time after the scanning of the object attribute. In one embodiment, a particularly high insensitivity toward relative movements between object and scanning device may also be achieved. A movement-insensitive and position-precise scanning device of this kind is well suited, but not limited to, mobile, portable applications.

In one embodiment, the scanning device uses a laser radiation source as the primary radiation source. The visualization device uses a laser display projector as the projector. The same laser radiation source is used for generating the primary radiation and the laser display projection. In this embodiment, a particularly simple and space-saving design is achieved. This is well suited, for example, to mobile-portable applications.

### BRIEF DESCRIPTION OF THE DRAWINGS

FIG. 1 shows one embodiment of a reflection scanning device with a visualization device,

FIG. 2 shows one embodiment of a transmission scanning device with a visualization device, and

FIG. 3 shows one embodiment of a scanning device with a visualization device with common laser radiation source

### DETAILED DESCRIPTION

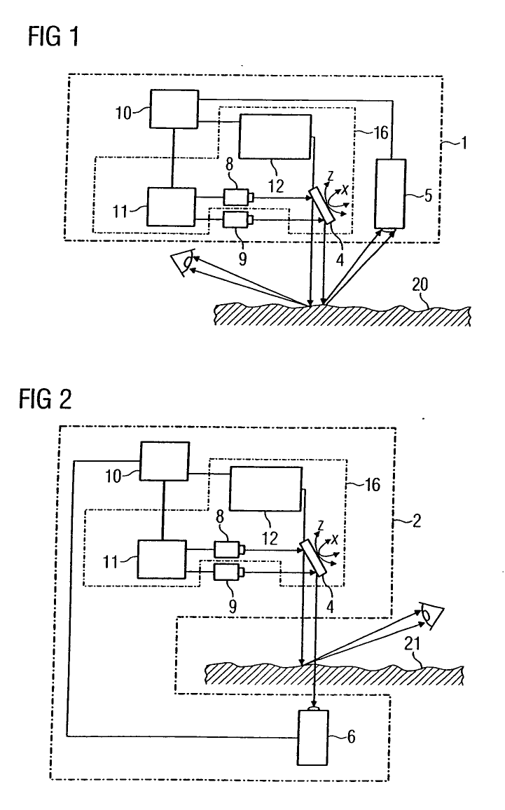

In one embodiment shown in FIG. 1, a laser controller 11 controls a laser source 9, which generates a primary beam. The laser source 9 may be embodied as a laser radiation source. The primary beam is illustrated in FIG. 1 as a line with an arrow in the direction of propagation. In one embodiment, primary beam includes a radiation beam. The primary beam is deflected by a deflection device, which includes a micromirror 4 and/or a prism. The micromiror 4 is controlled by a deflection controller 12. The deflected primary beam then strikes the surface of an object 20. The laser radiation source 9 may generate, for example, laser radiation in a wavelength range of 690 nm to 850 nm. In this embodiment, the scanning device 1 may be used in medical diagnosis of cancer for detecting fluorescence phenomena in tissue marked with a contrast agent.

In one embodiment, the object 20 reflects the primary beam and generates secondary radiation using this reflection. The secondary radiation is detected using a secondary radiation detector 5. The secondary radiation detector 5 is connected at the output end to the control device 10. The secondary radiation detector 5 may include, for example, a camera chip, a CCD, a photodiode, some other semiconductor detector, or any suitable detector.

In one embodiment, the control device 10 controls the laser controller 11 and the deflection controller 12. For example, at any given moment in time, position data relating to the point of the object 20 scanned in each case is present in the control device 10. In one embodiment, the control device 10 receives object attribute information of the scanned point of the object 20 from the secondary radiation detector 5.

In one embodiment, the control device 10 outputs the object attribute information and the position data to the deflection controller 12. The control device 10 outputs the object attribute information at the output end.

In one embodiment, the deflection controller 12 receives the object attribute information and/or the associated position data at the input end. The deflection controller 12 controls the micromirror 4 in such a way that a visualization of the object attribute information is projected in a position-directed manner onto the position identified by the position data. In this embodiment, a visualization of the object attribute information is projected onto the scanned point of the object 20. The projection is based on the laser beam coming from the laser radiation source 8. The laser radiation source 8 is controlled by the laser controller 11.

In one embodiment, a laser display projector includes the laser controller 11, a laser radiation source 3, the micromirror 4 and the deflection controller 12. In one embodiment, the visualization device 16 includes the laser controller 11, a laser radiation source 3, the micromirror 4 and the deflection controller 12. The deflection controller 12 receives the position data and object attribute information as input data from the control device 10.

In one embodiment, the visualization device 16 or the laser display projector includes a movable micromirror 4. The micromirror 4 may be rotated about two spatial axes, as indicated in the Figures by double arrows (z and x). In one embodiment, the visualization of the object attribute information is projected onto the object 20, where it is recognizable for an observer, as is indicated in the Figures by the schematically representation of an eye of an observer.

In one embodiment, as shown in FIG. 2, a scanning device 2 is operative to scan object attributes, which can be scanned by transmission. In one embodiment, the scanning device 2 includes a control device 10 and a visualization device 16. The visualization device 16 includes, as described in FIG. 1, a deflection controller 12, a laser controller 11 and/or a laser radiation source 8.

In one embodiment, the secondary radiation detector 6 lies on the other side of the object 21 when viewed from the perspective of the primary radiation source 9. For example, the secondary radiation detector 6 detects secondary radiation from a direction that is essentially the same as that of the primary radiation beam. In one embodiment, as shown in FIG. 1, the secondary radiation beam is detected from a direction that is substantially opposite to that of the primary radiation.

In one embodiment, object attribute information is received together with position data from the control device 10 and output to the deflection device 12, which controls the laser display projection in such a way that a visualization of the object attribute information is projected in a position-directed manner onto the position on the object 21 identified by the position data.

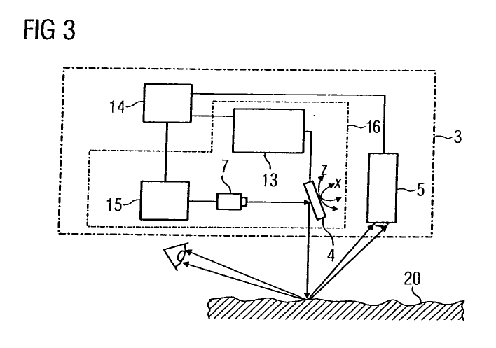

In one embodiment, as shown in FIG. 3, a scanning device 3 includes a laser controller 15, which controls a primary radiation source 7. The primary radiation source 7 generates a primary beam which is deflected by a micromirror 4, which is controlled by the deflection controller 13. The deflected primary beam strikes the surface of the object 20 and causes reflection.

In one embodiment, the reflected secondary radiation is detected by the secondary radiation detector 5. The scanning information is output at the output end to the control device 14. The control device 14 is connected to and controls both the laser controller 15 and the deflection controller 13. The control device 14 includes the position data of the point on the surface of the object 20 scanned in each case. In one embodiment, the control device 14 outputs the scanning information together with the object attribute information received from the secondary radiation detector 5 to the deflection controller 13. The control device 14 outputs information from the output end.

In one embodiment, the visualization device 16 includes the deflection controller 13, the laser controller 15, the laser radiation source 7 and/or the micromirror 4. The visualization device 16 projects a visualization of the object attribute information in a position-directed manner onto the point on the surface of the object 20 scanned in each case.

In one embodiment, the laser radiation source 7 serves both as the primary radiation source and as the laser display projection/laser radiation source. In this embodiment, the laser radiation source 7 fulfills a dual function.

Various embodiments described herein can be used alone or in combination with one another. The forgoing detailed description has described only a few of the many possible implementations of the present invention. For this reason, this detailed description is intended by way of illustration, and not by way of limitation. It is only the following claims, including all equivalents that are intended to define the scope of this invention.

1. A visualization device comprising:
a projector that is operative to-project in a position-directed manner;
a data input device that is operative to receive position data and associated position-specific object attribute information; and
a control device which is connected at the input end to the data input device and at the output end to the projector, wherein the control device is operative to control the projector to project a visualization of position-specific object attribute information received from the data input.

2. The visualization device as claimed in claim 1, wherein the projector comprises a laser display projector.

3. The visualization device as claimed in claim 2, wherein the laser display projector comprises a laser radiation source and a laser beam deflection device.

4. The visualization device as claimed in claim 1, further comprising a numeric or alphanumeric display.

5. The visualization device as claimed in claim 1, further comprising a graphical display of contours.

6. The visualization device as claimed in claim 1, further comprising a graphical display of areas.

7. A scanning device comprising:
a visualization device comprising a primary radiation source,
a secondary radiation detector, which is operative to detect secondary radiation generated by an object as a result of the incidence of primary radiation, and
an evaluation device, which is operative to determine position data and associated position-specific object attribute information as a function of a detection by the secondary radiation detector, wherein the evaluation device is operative to transmit the position data and associated position-specific object attribute information to a data input of the visualization device.

8. The scanning device as claimed in claim 7, wherein the visualization device comprises:
a projector that is operative to project in a position-directed manner;
a data input device that is operative to receive the position data and the associated position-specific object attribute information; and
a control device which is connected at the input end to the data input device and at the output end to the projector, wherein the control device is operative to control the projector to project a visualization of position-specific object attribute information received from the data input.

9. The scanning device as claimed in claim 7, wherein the primary radiation source comprises a laser radiation source.

10. The scanning device as claimed in claim 8, wherein laser radiation coming from the primary radiation source is deflected by a deflection device.

11. The scanning device as claimed claim 7, wherein the secondary radiation detector is operative to detect secondary radiation in a wavelength range from about 690 nm to 850 nm.

12. The scanning device as claimed in claim 7, wherein the object attribute information includes at least one of a contrast in the visible wavelength range, a contrast in the invisible wavelength range, a surface characteristic, an absorption behavior, a transparency, a fracture or a deposit.

13. The scanning device as claimed in claim 7, wherein the object attribute information includes a fluorescence.

14. The scanning device as claimed in claim 7, wherein the secondary radiation detector detects secondary radiation from a direction substantially opposite to that of the primary radiation.

15. The scanning device as claimed in claim 7, wherein the secondary radiation detector is operative to detect secondary radiation from a direction substantially the same as that of the primary radiation.

16. The scanning device as claimed in claim 7, wherein the scanning device is mobile and/or portable.

17. The scanning device as claimed in claim 9, wherein a laser radiation source is operative to be used simultaneously as the primary radiation source and a laser display projection.

18. The visualization device as claimed in claim 3, wherein the laser beam deflection device includes a movable micromirror or a movable prism.

19. The scanning device as claimed in claim 9, wherein the deflection device includes a movable micromirror, a movable prism or both.

20. A medical device for examining a tissue, comprising:
a scanning device including:
a visualization device;
a secondary radiation detector, which is operative to detect secondary radiation generated by an object, and
an evaluation device, which is operative to determine information as a function of a detection by the secondary radiation detector, wherein the evaluation device is operative to transmit the information to the visualization device.
